(19) United States
(12) Patent Application Publication
(10) Pub. No.: US 2011/0066080 A1
(43) Pub. Date: Mar. 17, 2011

(54) MEDICAL NAVIGATION METHOD & SYSTEM

(75) Inventor: Jan Nmi Stifter, Schneisingen (CH)

(73) Assignee: SMITH & NEPHEW ORTHOPAEDICS AG, Memphis, TN (US)

(21) Appl. No.: 12/988,040

(22) PCT Filed: Apr. 15, 2009
(86) PCT No.: PCT/EP09/02754

§ 371 Date: Nov. 19, 2010

(30) Foreign Application Priority Data
Apr. 15, 2008 (GB) 0806813.2

(51) Int. Cl. A61B 5/103

(52) U.S. Cl. 600/587

(57) ABSTRACT

A method of determining a rotational axis of a joint, comprising the steps of: moving at least one bone of the joint; recording coordinates of the at least one bone as it moves; and using the recorded coordinates to calculate a rotational axis. A system for determining a rotational axis of a joint, comprising: a device for moving at least one bone of the joint, in use; at least one fiducial for reversibly connecting to at least one bone, in use; at least one measurement system for recording coordinates of the at least one fiducial, in use; and at least one computer configured to execute the method of the present invention.

The present invention relates to a medical navigation method and system, particularly navigation methods and systems for determining the rotational axis of a joint. The joint may be a knee, elbow, ankle, toe, hip, finger or shoulder joint, for example.

In current orthopedic navigation systems, rotational axes are determined by the surgeon palpating with a pointer 2 points and the navigation system then calculates the virtual axes using these 2 points.

For example, in a Total Knee Replacement (TKR) surgery using a navigation system, known systems can palpate either the Whiteside line and/or the epicondylar line and/or the posterior line and calculate the rotational flexion axes out of the corresponding 2 landmarks. The surgeon has to select in the configuration of the navigation software the anatomical rotational landmarks he would like to use. For example, the surgeon may use either the Whiteside line, epicondylar line or the posterior condylar line. Some known systems have a method wherein all three of the above landmarks are displayed together on a suitable display means and the surgeon decides during the surgery which landmark to use.

However, this method is very imprecise, because the points are difficult to palpate and are close together (this is known as the “short axis problem”). Consequently, a small error in palpation results in a significant lack of precision. In fact, all known navigation systems yield imprecise rotational axes calculations [Matziolis, Orthopäde 2006•35:848-852, Siston, JBJS 2005; 87:2276-2280]. This is because there is no reliable landmark that can be precisely palpated. Accordingly, a small palpation error results in a significant error in the orientation of the implant.

Another disadvantage of known TKR navigation systems is that if the knee has bony defects, which is usually the case in arthritic knees, the points are measured on the bone with defects. Accordingly, the defect leads to an imprecise point measurement and hence the axis is determined at the wrong position, leading to malrotations.

Accordingly, the present invention overcomes the above disadvantages and problems.

According to a first aspect of the present invention, there is provided a method of determining a rotational axis of a joint, comprising the steps of:
moving at least one bone of the joint;
recording coordinates of the at least one bone as it moves; and
using the recorded coordinates to calculate a rotational axis.

In the present application, the term rotational axis means the same as a kinematic axis (kinematically determined axis) or a functional kinematic axis or a flexion axis.

According to a second aspect of the present invention, there is provided a method of determining a rotational axis of a joint, comprising the steps of:
attaching at least one fiducial to at least one bone of the joint;
moving the at least one bone between a first position and a second position;
recording coordinates of the at least one fiducial with a measurement system whilst moving the bones between the first and the second position; and
using the recorded coordinates to calculate a rotational axis.

In the present application, the term fiducial means any device whose coordinates are capable of being recorded by a measurement/navigational system. A fiducial may be an array, a rigid body, or a locator, for example.

The or each bone may be moved in such a way that it stretches the ligaments. That is, the movement may take the slack out of the ligaments.

The joint may comprise two bones and the bones may be moved relative to each other. For example, the joint may be a knee joint, elbow joint, ankle joint, finger, hip, shoulder or toe joint.

The calculation of the rotational axis may be performed by an algorithm.

According to embodiments of the present invention, determination of at least one landmark on the at least one bone may be determined prior to calculation of the rotational axis.

In the present application, a landmark may be the result of point acquisition, point measurement, or point definition, and/or may be determined by an algorithm. The or each landmark may be determined using a technique such as computer assisted surgery (CAS), computerised tomography (CT), magnetic resonance imaging (MRI), ultrasound, or X-ray analysis, or a combination of any such techniques.

The algorithm may comprise transformation of the or each landmark determined prior to calculation of the rotational axis using the recorded coordinates.

The algorithm may comprise elimination of non-relevant recorded coordinates.

The algorithm may comprise transformation of the or each landmark determined prior to calculation of the rotational axis using the recorded coordinates into the coordinate system of the other bone for all positions between the first and second position.

The algorithm may comprise a best fit calculation of a plane based on the or each transformed landmark.

The best fit calculation may comprise least squares (least distance) analysis.

The algorithm may comprise the determination of a perpendicular axis to the plane. The normal to the plane is the rotational axis.

The at least one bone may be moved by mechanical, hydraulic or pneumatic means.

The mechanical means may comprise a spreading device. For example, the mechanical means may comprise a spreading device such as that disclosed in WO 2007/045460.

The hydraulic means may comprise a biocompatible fluid, for example physiologic water or saline.

The hydraulic means may comprise a biocompatible fluid contained in a receptacle such as a balloon.

The pneumatic means may comprise a balloon. The balloon may be inflated or deflated by the addition or removal of air, respectively.

According to a third aspect of the present invention, there is provided a system for determining a rotational axis of a joint, comprising:
a device for moving at least one bone of the joint, in use;
at least one fiducial for reversibly connecting to at least one bone, in use;
at least one measurement system for recording coordinates of the at least one fiducial, in use; and
at least one computer configured to execute the method according to the first or second aspects of the present invention.

According to preferred embodiments of the present invention, the method uses a spreading device to spread two bones apart, so that the ligaments of the joint are stretched. A fiducial/array is reversibly connected to each of the two bones. The joint is then moved slowly from one position into another position. A measurement/navigation system records the coordinates of these two fiducials during this movement. The fiducials are measured with a measurement system (optical, electromagnetic, etc.) and the recorded coordinates are processed with a computer. The computer uses a novel algorithm to calculate a plurality of axes from the measurements and then a rotational axis (kinematic flexion axis) of the joint is calculated. As an alternative to the spreading device, the plurality of axes may be calculated using externally applied forces, for example by applying forces to the ankle.

The present invention overcomes the problems of the prior art and has a number of advantages. The present invention does not use bony defects and therefore avoids the problems discussed above. The present invention does not rely on short axes and avoids palpation errors of the surgeon and/or measurement errors. The present invention takes into account the ligaments of the knee, so that a functional, kinematic axis can be determined, instead of a static axis determined from bony landmarks. The present invention works with deformed ligaments. The present invention provides an easy procedure during surgery without relying on anything more complicated than a spreading device or other means described herein.

The calculated rotational axis has a number of uses. It can be used to align the surgical cuts, so that the implant is rotated according to the kinematics of the joint. It can be used for positioning of an implant to the optimal functional position for a particular joint. Since the axis is in between two bones, it can be used to align implants and surgical cuts for both bones, not only for one bone.

Embodiments of the present invention may be used in TKR using a navigational system such as the Smith & Nephew PiGalileo system. The surgeon places a fiducial on the femur and a fiducial on the tibia and the relevant landmarks are palpated by the surgeon.

Landmarks may be specific anatomical points, areas, regions that are determined by the surgeon using the methods described above, such as palpation with a pointer, stylus, ultrasound, MRI, CT, X-Ray, automatic detection by an algorithm, etc.

The landmarks may be measured by the system when a footswitch is pressed by the surgeon, for example. Alternative systems may have different user interaction/control than a footswitch. Other possible user interfaces include: special movements of a pointer detected by the system, voice control, remote control, optical panel, a stylus pointed at a display screen, etc.

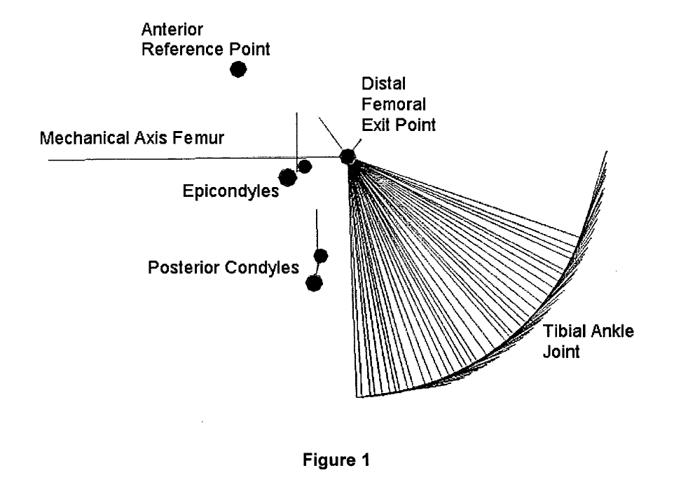

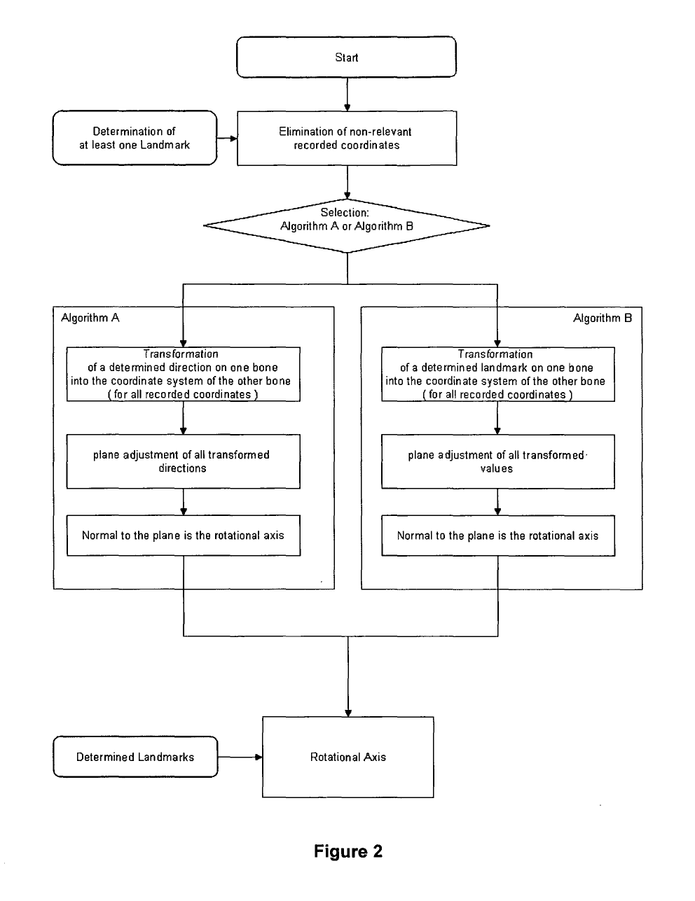

After all the relevant landmarks are acquired by the system, a tibial cutting jig is navigated and oriented according to the values on the screen and then the tibia is cut. A mechanical spreader such as that disclosed in WO 2007/045460 is placed and secured with a pin to prevent it from sliding out of the knee joint. The spreader is released to force the knee apart. The capsule may be closed to more precisely measure the kinematics. The knee is slowly bent from flexion to extension. The navigation system measures the bending and a plurality of axes (see FIG. 1) is calculated by an algorithm. From these axes, one precise axis can be evaluated by the algorithm. FIG. 2 shows two algorithms (A and B) which may be utilised in accordance with the present invention.

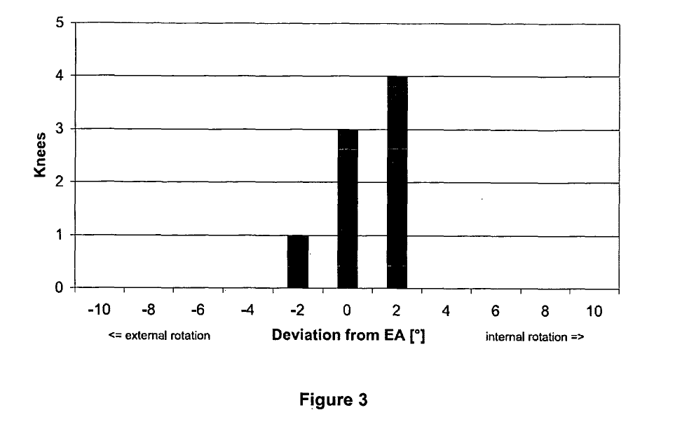

An algorithm according to the present invention has been evaluated in a clinical trial. As shown in FIG. 3, the applied algorithm yields precise results when compared to the bony epicondylar axis (EA). The identified knee joint axis values show only a slight variance within the given range of movement. There is a close relationship between the knee joint axis and the epicondylar axis defined by computer tomography, regardless of any preoperative malalignment compared to the surgical epicondylar axis. The results show that the applied algorithm may be used to determine a functional knee joint axis, and that the outcome is reproducible.

What is claimed is:

1. A method of determining a rotational axis of a joint, comprising the steps of: moving at least one bone of the joint; recording coordinates of the at least one bone as it moves; and using the recorded coordinates to calculate a rotational axis.

2. A method of determining a rotational axis of a joint, comprising the steps of: attaching at least one fiducial to at least one bone of the joint; moving the at least one bone between a first position and a second position; recording coordinates of the at least one fiducial with a measurement system whilst moving the bones between the first and the second position; and using the recorded coordinates to calculate a rotational axis.

3. A method according to claim 1, wherein the at least one bone is moved in such a way that it stretches the ligaments.

4. A method according to claim 1, wherein the joint comprises two bones and the bones are moved relative to each other.

5. A method according to claim 1, wherein the calculation of the rotational axis is performed by an algorithm.

6. A method according to claim 1, wherein determination of at least one landmark on the at least one bone is determined prior to calculation of the rotational axis.

7. A method according to claim 6, wherein the at least one landmark is determined using a technique selected from the group consisting of CAS₁ CT, MRI, ultrasound, and X-ray analysis.

8. A method according to claim 6, wherein the algorithm comprises transformation of the at least one landmark determined prior to calculation of the rotational axis using the recorded coordinates.

9. A method according to claim 5, wherein the algorithm comprises elimination of non-relevant recorded coordinates.

10. A method according to claim 5 when dependent on claim 2, wherein the algorithm comprises transformation of the at least one landmark determined prior to calculation of the rotational axis using the recorded coordinates into the coordinate system of the other bone for all positions between the first and second position.

11. A method according to claim 5, wherein the algorithm comprises a best fit calculation of a plane based on the transformed at least one landmark.

12. A method according to claim 11, wherein the best fit calculation comprises least squares analysis.

13. A method according to claim 11, wherein the algorithm comprises the determination of a perpendicular axis to the plane.

14. A method according to claim 1, wherein the at least one bone is moved by mechanical, hydraulic or pneumatic means.

15. A method according to claim 14, wherein the mechanical means comprises a spreading device.

16. A method according to claim 14, wherein the hydraulic means comprises a biocompatible fluid.

17. A method according to claim 14, wherein the pneumatic means comprises a balloon.

18. A system for determining a rotational axis of a joint, comprising: a device for moving at least one bone of the joint, in use; at least one fiducial for reversibly connecting to at least one bone, in use; at least one measurement system for recording coordinates of the at least one fiducial, in use; and at least one computer configured to execute steps of recording coordinates of at least one bone as it moves and using the recorded coordinates to calculate a rotational axes.

19. (canceled)

20. (canceled)
